(19) United States
(12) Patent Application Publication
(10) Pub. No.: US 2010/0087711 A1
(43) Pub. Date: Apr. 8, 2010

(54) Repeatably flexible surgical instrument

(75) Inventor: Kevin C. Edwards, Olive Branch, MS (US)

Correspondence Address: OLIFF & BERRIDGE, PLC, P.O. BOX 320850, ALEXANDRIA, VA, 22320-4850, US

(73) Assignee: GYRUS ENT, L.L.C., BARTLETT, TN (US)

(21) Appl. No.: 12/585,779

(22) Filed: Sep. 24, 2009

Related U.S. Application Data
(60) Provisional application No. 61/136,817, filed on Oct. 6, 2008.

(51) Int. Cl. A61B 1/00

(52) U.S. Cl. 600/139

(57) ABSTRACT

A flexible-shaft surgical instrument is provided that can be repeatably bent to one or more desired angles on demand by a user without the use of a special bending tool. The flexible-shaft surgical instrument can be repeatably bent and re-bent into multiple positions without plastic deformation of the flexible-shaft surgical instrument occurring in any of one or more bend portions.

This application claims the benefit of U.S. Provisional Patent Application No. 61/136,817, filed Oct. 6, 2008.

### BACKGROUND

This disclosure relates to an improved surgical instrument structure for surgical instruments such as shavers and microdebriders.

Surgical instruments with thin, elongated shafts for accessing various surgical sites through natural openings in the body, or through surgically-inserted cannulae, are known. These surgical instruments may be provided with generally thin, elongated shafts in either straight or curved configurations. An illustrative example of a curved shaft surgical instrument is the curved-shaft shaver blade surgical instrument disclosed in U.S. Pat. No. 4,646,738 to Trott.

In thin elongated-shaft surgical instruments, it is often useful, or even necessary, for a surgeon to be able to precisely orient the instruments' distal end with respect to the target site in the patient's body during surgery. This is a relatively straightforward procedure with straight-shaft surgical instruments. This procedure, however, tends to become more complicated with curved-shaft surgical instruments. An example of a curved-shaft surgical instrument is the curved-shaft shaver blade surgical instrument, specifically adapted so that the cutting window located proximally adjacent its distal end can be reasonably easily re-oriented, is provided in U.S. Pat. No. 5,411,514 to Fucci et al.

Variations to the rigid straight-shaft or curved-shaft surgical instruments, such as the shaver blades discussed briefly above, have been introduced. Flexible-shaft surgical instruments have been developed. In such devices, generally, a powered hand piece drives a flexible inner surgical member, such as an inner cutting member in a flexible-shaft shaver blade surgical instrument. The flexible inner surgical member is generally housed and supported in a semi-rigid outer elongated thin shaft. The semi-rigid outer elongated thin shaft generally provided with at least one bendable portion. The at least one bendable portion differentiates conventional flexible-shaft surgical instruments from conventional rigid curved-shaft surgical instruments. Conventional flexible-shaft surgical instruments often include outer shafts members in which the bendable portion is a single continuous tube having, for example, a ribbed portion with alternating thick and thin wall thicknesses along the length of the shaft in a specific bend region. This allows for the outer shaft to be bent more easily than a non-ribbed conventional outer shaft member with a constant wall thickness in its bend region. Such construction is intended to reduce, for example, crimping in the bendable portion that could impact the inner flexible surgical member rendering it partially or wholly inoperable.

A number of difficulties have been encountered in attempting to develop reliable flexible-shaft surgical instruments, particularly in developing such instruments that may be repeatedly bendable. To reduce the frequency of a possibility of crimping, for example, some flexible-shaft surgical instruments require a compatible bending tool to provide a user with a controlled method for bending the device to a desired angle according to a specific bend radius. Without the use of such a bending tool, the flexible shafts of the devices are subject to the above-mentioned crimping through user error resulting in the outer shafts of the surgical instruments being bent, for example, at too great an angle or according to too small a bend radius.

Other problems with conventional flexible-shaft surgical instruments include that, even if not crimped, they are often subject to some plastic deformation, once bent, leading to limited reusability. If bends are not controlled, or bends are made too often, the tubes of the conventional flexible-shaft surgical instrument may kink, crimp, collapse, rupture or otherwise fail. This plastic deformation may also negatively affect the function of the inner surgical instrument such as the cutting member on a first or subsequent uses.

An additional drawback to a conventional flexible-shaft surgical instrument is that the plastic deformation that occurs in any of the tubes prevents the device from being bent in different planes successfully and successively. Once a tube is bent one time in a conventional flexible-shaft surgical instrument, it may never regain its original shape. Therefore, conventional flexible-shaft surgical instruments are limited to a finite set of orientations, e.g. concave, convex, left, right, and bend angle combinations, as well as minimal, if any, re-use.

### SUMMARY

User preferences, such as those of surgeons, modified by their patients' needs, dictate limitless combinations of bend parameters with regard, for example, to angles of the bend and window positions, in the case of flexible-shaft surgical instruments. Accommodating such user preferences, and the full scope of patient anatomies, during surgical procedures is often difficult with the use of conventional flexible-shaft surgical instruments. Ease of bending a flexible-shaft surgical instrument, as well as enabling a surgeon to adapt on the fly in a surgery, is also difficult with a conventional flexible-shaft surgical instrument, particularly one that plastically deforms when bent.

Additional restrictions on flexible-shaft surgical instruments include that they are required to be formed of materials that are biocompatible. Further, if the flexible-shaft surgical instruments are to be re-used, the materials from which they are formed need to be able to withstand the rigors of repeated cleaning and sterilization.

It would be advantageous to provide a flexible-shaft surgical instrument that can be bent to a desired angle, or combination of desired angles in more than one plane, on demand by a user. To any extent that such flexible-shaft surgical instruments can be bent without the use of a special bending tool, provides an additional advantage to the user. Further, a flexible-shaft surgical instrument that can be repeatedly bent and re-bent into multiple positions without plastic deformation of the flexible-shaft surgical instrument occurring in any of its bend portions is also highly desirable.

Considerations in the design and development of each surgical instrument include the following. The surgical instrument must be made of biocompatible materials that can be sterilized after each use without affecting necessary compatibilities or bending capacity, including a capacity to be bent along multiple axes singly, or in a compound manner. The materials must be assembled such that the device is able to be easily deformed yet retain its deformed shape, once manipulated, without returning to a pre-deformed shape. In other words, though flexible, the shaft of the instrument must be sufficiently rigid in its bend portion such that, once deformed, the flexible shaft of the surgical instrument will retain its deformed shape according to the user's formation of the shaft. The shaft may be formed from wear resistant and heat resistant materials. The materials are preferably easily manufactured and, may be non-conductive. The materials should be selected to avoid other problems with particular materials such as brittleness, lack of flexibility and poor machinability.

It would be advantageous to provide a flexible-shaft surgical instrument capable of being easily manipulated by surgeon control without the traditional drawbacks of a conventional flexible-shaft surgical instrument, discussed above. Such a device could operate as a “one-size-fits-most” single-device solution to using multiple devices, and could allow customization of the bend parameters for the surgeon user.

It would be advantageous to provide a flexible-shaft surgical instrument that may be repeatably bendable in a manner particularly where the bend portion is wear resistant to prevent degradation of a semi-rigid bend portion and enable re-use of the device. The flexible-shaft surgical instrument may be formed of a creep-resistant material such that the deformation characteristics of the device are not negatively affected over the course of its shelf life. The flexible shaft surgical instrument may be formed of a material that is additionally temperature resistant so that the flexible-shaft surgical instrument may survive multiple sterilization procedures to allow the flexible-shaft surgical instrument to be used multiple times. The flexible-shaft surgical instrument may be formed from a material that is non-conductive to prevent electrical shorts between the flexible-shaft surgical instrument, and other surgical instruments that are used in cooperation with the flexible-shaft surgical instrument. The flexible-shaft surgical instrument may also be formed of a material that is non-reactive with most chemicals encountered in a surgical procedure in which the flexible-shaft surgical instrument may be used.

In various exemplary embodiments, a flexible-shaft surgical instrument may be provided that includes at least one semi-rigid shaft portion. The at least one semi-rigid shaft portion may advantageously incorporate a wound structure to make a flexible, but rigid, tube portion that is a repeatedly bendable structure. This wound structure may enable a surgeon to control the degree of bend of the semi-rigid shaft portion and enable the surgeon to return the instrument to a straight orientation to be later re-bent and re-used without plastic deformation. The wound structure is preferably formed by interleaving at least two flexible elements with one another. The two flexible elements may include spring-like structures with the cross-sectional shape of the material comprising the spring-like structures being the same or different. They may also be formed from same or different materials.

Devices according to this disclosure may include surgical instruments having repeatably flexible outer shaft portions in order to accommodate a wide array of internal surgical components. Devices according to this disclosure may provide surgical instruments having outer portions that have an opening at or near a distal end such as a cutting window or a portal to otherwise accommodate any manner of surgical instrument. These surgical instruments include, but are not limited to, a surgical light, camera or other observation device, or some manner of surgical instrument tip for cutting, cauterizing or otherwise treating a surgical site within a patient. A cutting window, in a shaver blade instrument, for example, within an outer portion may allow for engagement between a patient's tissue and a cutting element in the inner portion.

In various exemplary embodiments, the flexible-shaft surgical instrument should not require the use of a bending tool to make a “correct” bend. The flexible-shaft surgical instrument may be bendable by a multitude of techniques and according to surgeon user preferences. The flexible-shaft surgical instrument may also be bent without plastic deformation to the bendable portion that takes place when the flexible-shaft surgical instrument is bent. Therefore, a semi-rigid shaft of the flexible-shaft surgical instrument may be bent multiple times in multiple planes or may otherwise be returned to a substantially straight orientation after bending. The reliability of such a device is not compromised when the semi-rigid shaft is bent, and its life expectancy may be increased when compared to conventional flexible-shaft surgical instruments.

In various exemplary embodiments, the device may be scalable, allowing flexible-shaft surgical instruments to be assembled which may be directed to differing patient anatomies (such as children and adult sizes), and to specific operating procedural requirements (e.g., nasopharyngeal or sinus procedures).

In various exemplary embodiments, the device may also have electrosurgical components added to the flexible-shaft surgical instrument using similar assembly materials and methods existing in known flexible-shaft surgical instruments.

In various exemplary embodiments, the flexible-shaft surgical instrument may include a wound portion, described in paragraph [0014] above, that is formed from wear-resistant and biocompatible polymer compounds specifically adapted to such use.

In various exemplary embodiments, the flexible-shaft surgical instrument may also have one or more rigid shaft members that extend from either side of the at least one semi-rigid portion, and also may incorporate a protective sheath to protect at least one semi-rigid portion, as well as any rigid portions of the flexible-shaft surgical instrument.

In various exemplary embodiments, the flexible-shaft surgical instrument may be a flexible shaver blade having a cutting window and blade portion for cutting a patient's tissue. However, the flexible-shaft surgical instrument may take other forms such as an endoscope, light, camera, vacuum, suction lumen, electrosurgical instrument and the like.

These and other features and advantages of the disclosed device are described in, or apparent from, the following detailed description of various exemplary embodiments.

### BRIEF DESCRIPTION OF THE DRAWINGS

Various exemplary embodiments of the disclosed flexible-shaft surgical instrument will be described, in detail, with reference to the following drawings wherein:

FIG. 1 illustrates a perspective view of an exemplary flexible-shaft surgical instrument that incorporates a semi-rigid portion according to the disclosure;

FIG. 2 illustrates a perspective view of an exemplary semi-rigid portion of a flexible-shaft surgical instrument including a wound portion according to the disclosure;

FIG. 3 illustrates a perspective view of an exemplary flexible-shaft surgical instrument that is a flexible shaver blade having a protective sheath according to this disclosure;

FIG. 4 illustrates a perspective view of an exemplary flexible-shaft surgical instrument that is a flexible shaver blade illustrating a wound portion as the semi-rigid portion;

FIG. 5 illustrates a perspective view of a second exemplary flexible-shaft surgical instrument including a wound portion as its semi-rigid portion that varies and is interchangeable; and

FIG. 6 illustrates a perspective view of a third exemplary flexible-shaft surgical instrument that incorporates two semi-rigid portions.

FIG. 7 illustrates a perspective view of a cross-section of an exemplary semi-rigid portion according to this disclosure.

FIG. 8 illustrates a perspective view of a cross-section of an exemplary semi-rigid portion in a bent orientation according to this disclosure.

### DETAILED DESCRIPTION OF EMBODIMENTS

The following embodiments illustrate examples of a flexible-shaft surgical instrument that may be bent to a desired angle on demand by a user without the use of a special bending tool. Disclosed embodiments of the flexible-shaft surgical instrument may be repeatably bent and re-bent into multiple positions without plastic deformation of the flexible-shaft surgical instrument occurring in any of its bend portions. While the disclosed embodiments may refer specifically to a repeatably bendable surgical instrument such as a shaver blade surgical instrument, this example is provided only as being illustrative of a surgical instrument which may gain special advantage based on the repeatably bendable configuration of a semi-rigid shaft portion according to this disclosure. It should be recognized, however, that a device including a semi-rigid shaft portion according to this disclosure may find utility in supporting any manner of surgical instrument where, for example, access is gained to a target surgical site inside a patient's body via one or more natural openings in the patient's body and/or via one or more surgically-inserted cannulae. In this regard, specific disclosed examples of surgical instruments, and the use of specific terms to describe those instruments, should be considered as illustrative only, and not limiting.

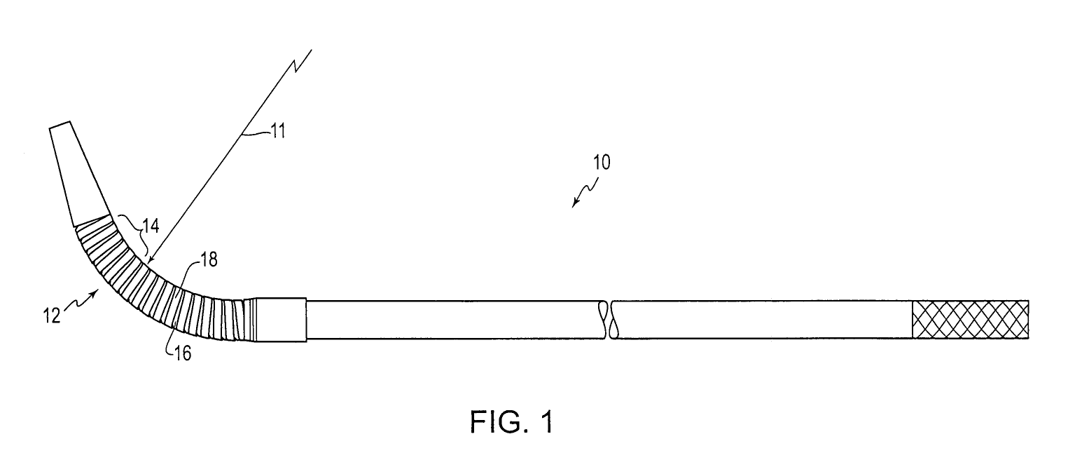

FIG. 1 illustrates a perspective view of an exemplary flexible-shaft surgical instrument 10 that incorporates a semi-rigid portion 12. The semi-rigid portion 12 in this exemplary embodiment comprises a wound portion 14 that forms a repeatably bendable structure. The wound portion 14 may be formed from a biocompatible polymer material or metal. The wound portion 14 comprises at least two helically wound elements 16 and 18, that may be formed from a same or a different material, interleaved with one another.

Flexible-shaft surgical instruments traditionally have a center line radius 11 which determines the extent that the semi-rigid portion 12 may be bent. Depending on the relative thickness, and other geometries of the first element 16 and the second element 18, the center line radius 11 can range from 0.25 to two inches. The semi-rigid portion 12, again depending on the relative thickness of the first element 16 and the second element 18, may be bendable between angles ranging from 0° to 275°. The semi-rigid portion 12 may be bendable in different planes, at the same time, or successively. Various arrangements of the wound portion of the flexible-shaft surgical instrument may have varying holding strengths between particular portions of the semi-rigid portion. For instance, the relative thickness of adjacent portions of the first element 16 and the second element 18 could vary from one end of the semi-rigid portion to the other end to afford variable bend radii and holding strengths along the length of the semi-rigid portion 12.

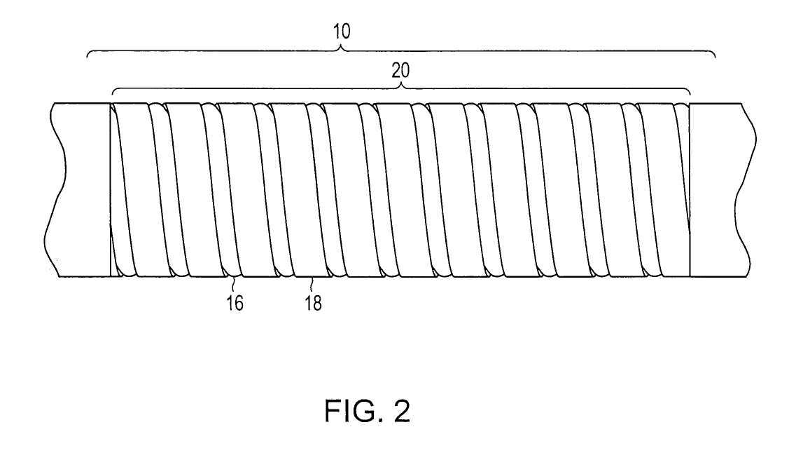

FIG. 2 illustrates a perspective view of wound portion 20 that makes up a semi-rigid portion 12 of a flexible-shaft surgical instrument 10.

A cross-sectional thickness of each of the first and second elements 16 and 18 that make up the wound portion 20 should be such that, when interleaved, an inner diameter of the semi-rigid portion 12 formed from the wound portion 20 remains constant whether it is in a straight or bent orientation. The particular cross-sectional thickness of the first and second elements 16 and 18 provide structural support such that the semi-rigid portion 12 does not collapse into itself when bent. Otherwise, the semi-rigid portion 12 may collapse in one axis of its cross section when bent. This collapse may squeeze a surgical device that is inside of the flexible-shaft surgical instrument, such as a flexible shaver, light or camera, and negatively affect the performance of the device.

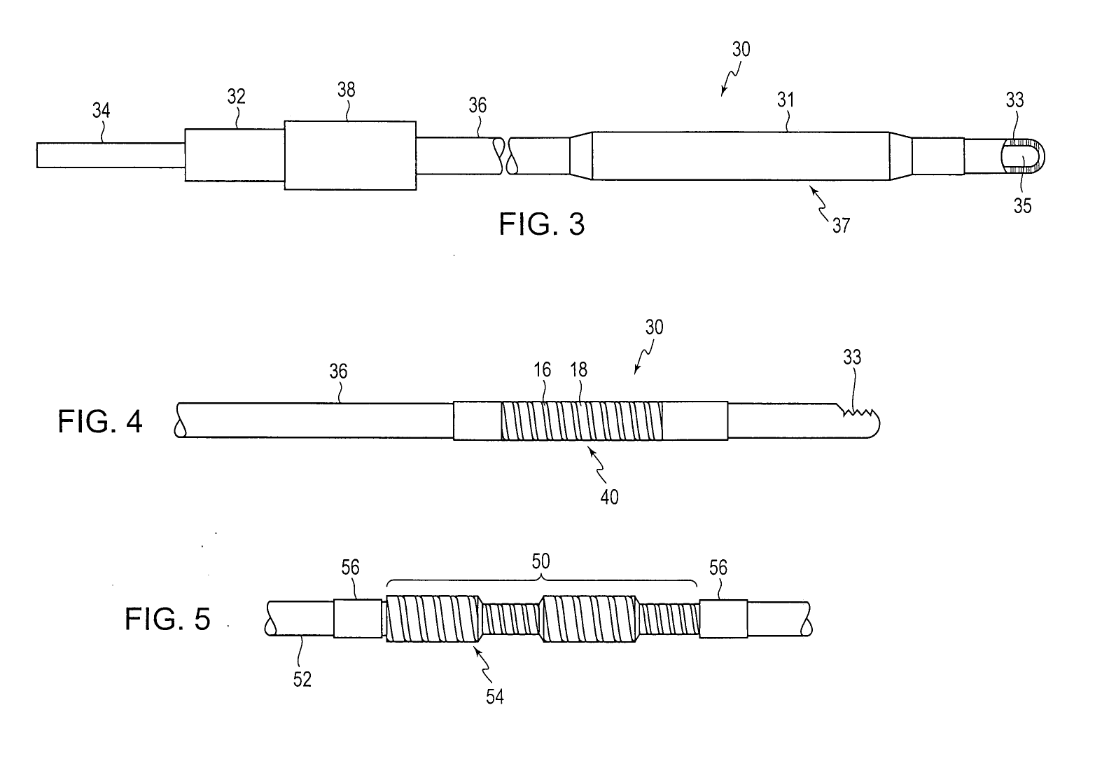

FIG. 3 illustrates a perspective view of a flexible-shaft surgical instrument that is a flexible shaver blade 30 that comprises an inner tube 34 that has a proximal end and a distal end. The inner tube 34 has a cutting element adjacent to its distal end. The flexible-shaft surgical instrument further comprises a first hub 32 that is connected to the proximal end of the inner tube 34 and allows the inner tube 34 to be driven for rotation along a central axis. The flexible shaver blade 30 further comprises an outer tube 36 that has a second hub 38. The inside diameter of the outer tube 36 is larger than an outside diameter of the inner tube 34. The outer tube 36 comprises a proximal portion attached to the second hub 38 and a semi-rigid portion 31 that has a proximal end that is attached to the proximal portion of the outer tube 36. The outer tube 36 has a distal portion that is attached to a distal end of the semi-rigid portion 31 and provides for a cutting window 35 at, or in the vicinity of, the extreme distal end of the instrument. The distal portion of the outer tube 36 provides for the cutting window 35 to face a predetermined direction and enables the cutting element 33 to cut tissue. The semi-rigid portion 31 comprises at least one wound portion 40 as shown in FIG. 4 that has a first element interleaved with a second element such that the semi-rigid portion may be repeatably bent to any predetermined angle and returned to a pre-bent orientation by a user while maintaining the structural integrity of the semi-rigid portion 31. In the configuration depicted in FIG. 3, the semi-rigid portion includes a protective sheath 37.

The wound portion 40, shown in FIG. 4, may be formed from various polymers or metals, or any combination thereof, such as, for example, a polymer coated metal or some other type of coated combination of metals. These particular polymers or metals are usable in the medical device field because they are biocompatible. These polymers or metals are also very heat resistant and exhibit reasonable structural strength and resilience. The wound portion 40 may also be molded, cast, milled, modified, or otherwise formed of other materials.

Examples of biocompatible materials include, for example as discussed above, various metals, polymers, or the like. Such materials are approved for use in medical devices. Additional advantages are that these materials can be more easily formed to desired structures by varying processes such as machining, or injection molding, extrusion or manual forming into helical elements, than other bio-compatible materials such as certain metals that are not easily formed into desired structures. These materials may also be non-conductive, have relatively high strength, are elastically expandable, and elastically bendable, like a spring, without fracturing or plastic deformation, have high wear resistance, and are rated for high temperature use, making them autoclavable. Rating for high temperature use is important so that the flexible-shaft surgical instrument may survive multiple sterilizations and be re-used a plurality of times without adversely affecting the structural integrity of the bendable portions.

It is particularly advantageous if a material is also creep resistant. Creep is an inherent condition of certain plastics and polymers where the strength of the material is gradually lost over time if the material is repeatedly or consistently exposed to a loading or bending force. Loading and bending forces would be present in exemplary embodiments such as those described here including a helically wound structure. Some typical biocompatible polymers are less creep resistant and would thus have a shorter shelf life if used according to the exemplary embodiments. Because a material that may be used for the wound portion 40 may be creep resistant, the shelf life may exceed that of typical biocompatible polymers if used according to the exemplary embodiments. When assembled, one of the elements of the wound portion 40 is under a load condition with respect to the other wound element that exhibits stresses on the assembled parts which may tend to promote deformation from creeping. This relationship between elements gives the assembled device the requisite stiffness in the bent condition. It may also be advantageous to form the wound portion 40 from a material that is also non-reactive to most chemicals found in surgical procedures.

The wound portion 40 may be permanently connected through means such as welding or detachably connected to the outer tube 36 so that the semi-rigid portion 31 may be interchangeable with other configurations of semi-rigid portions. The wound portion 40 governs the bendability of the semi-rigid portion 31.

The degree of bend of the semi-rigid portion may be dependent upon the relative size, temper, and/or stiffness of a first and second element of the wound portion 40 to one another, as well as the amount of friction that these elements experience between one another. The size of the wound portion 40 may also determine the amount of force that is required to bend the semi-rigid portion 31 to a desired bend radius, and the resistance of the device to move while in use, based, for example, on the amount of frictional surface area between the helically wound elements of the wound portion 40 at their points of contact.

In some applications, a surgeon, for instance, may wish to bend the semi-rigid portion 31 in a particular arrangement and hold that arrangement during use. In other applications, the surgeon may wish to have a more flexible feed as he manipulates the flexible-shaft surgical instrument into position. The flexible shaver blade 30, therefore, may be manipulated with minimal force.

The flexible shaver blade 30 may have a protective sheath 37 that covers at least the semi-rigid portion 31, but may cover the entire outer tube 36. The protective sheath 35 may be desirable to keep the wound portion 40 free from any debris that may inhibit its functionality or shorten its life expectancy. The sheath may also be used to provide hermeticity of the flexible shaver blade 30. The sheath may also be used as insulation over the proximal 36 and distal outer tube 36 when incorporating electrosurgical components to the device.

FIG. 5 illustrates a perspective view of a semi-rigid portion 50 of a flexible-shaft surgical instrument 52 that has wound portion 54 varies in size from one end to another, and is interchangeable. The semi-rigid portion 50 has a collar 56 at each end that facilities the interchangeability of the semi-rigid portion 50 into and out of the flexible-shaft surgical instrument 52.

Various embodiments may include different sized wound portion 54 to account for varying bend radii, as well as varying holding strengths. The force at which the semi-rigid portion 50 may be bent is dependent upon the particular overall diameter of the wound portion 54, and the size, shape, separation between sections of each element, and material properties of the respective elements. The amount of friction along the shaft may be equal or variable across a plurality of helically wound elements 56 that make up the wound portion 54 that makes up the semi-rigid portion 50, depending on the particular sizes and shapes of adjacent surfaces between each helically wound element.

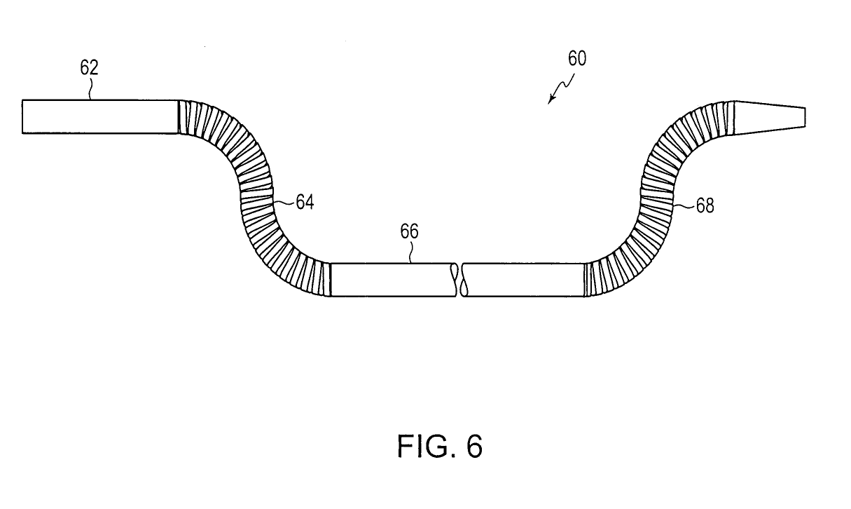

FIG. 6 illustrates a perspective view of a flexible-shaft surgical instrument 60 having a first rigid portion 62 connected to a first semi-rigid portion 64, a second rigid portion 66 that is connected to the first semi-rigid portion 62 and a second semi-rigid portion 68. The first and second semi-rigid portions 64 and 68 enable the first and second rigid portions 62 and 66 to be orientable in the same or varying planes.

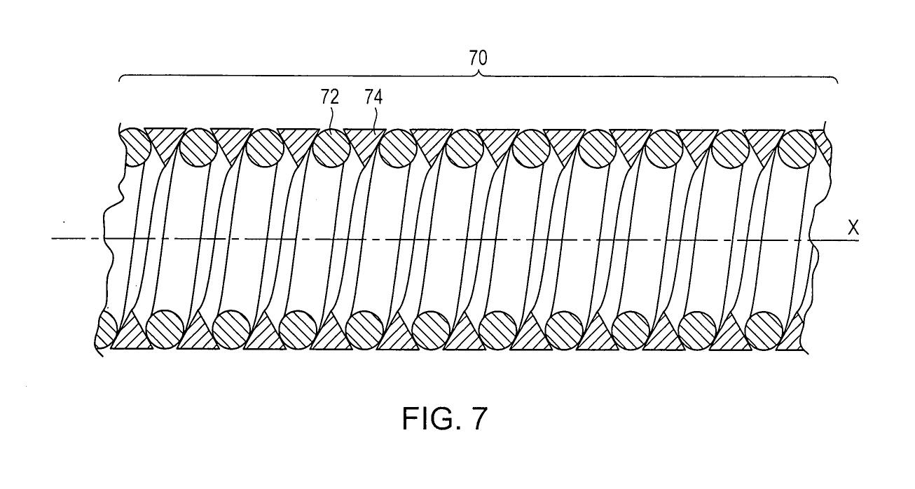

As illustrated in FIG. 7, a cross-sectional view of the semi-rigid portion 70 formed from helically wound elements provides that a first element 72 has a circular cross-section and a second element 74, that is adjacent to the first element 72, has a triangular cross-section. The first and second elements may be of a same or a different shape, and may be of any shape. The illustrated circular and triangular cross-section 5 are merely exemplary embodiments. Particularly, at least one of the elements may act as a wedge with the other element.

The variation in shapes between adjacent portions of the first element 72 and the second element 74 may create frictional resistance between the first element 72 and the second element 74 such that the semi-rigid portion 70 may be bent and hold its bend at a predetermined position.

As an example, if the first interleaved element 72 has a circular cross-section and the second interleaved element 74 has a triangular cross-section, the circular-shaped first element 72 will always be in contact with the triangular-shaped second element 74 when the semi-rigid portion 70 is bent and re-bent. By remaining in contact, there is an opportunity for increased frictional resistance between any adjacent surfaces of the two interleaved first and second elements 72 and 74 based on their cooperating structures.

The above-mentioned relationships between adjacent portions of the interleaved first and second elements 72 and 74 facilitates in maintaining the reliability of the form of the semi-rigid portion 70 without negatively affecting the repeatably bendable reliability of the devise. This is so because the relationship between the first and second elements 72 and 74 keeps these elements in contact, and within a particular tolerance, so that the semi-rigid portion may not be over-bent or become separated. Therefore, plastic deformation of the device is avoided. Further, the amount of friction that is experienced between adjacent portions of the first and second interleaved elements 72 and 74 holds the semi-rigid portion 70 at a predetermined bend orientation because the force of friction between these elements exceeds that of any opposing force created by either of the interleaved first and second elements 72 and 74.

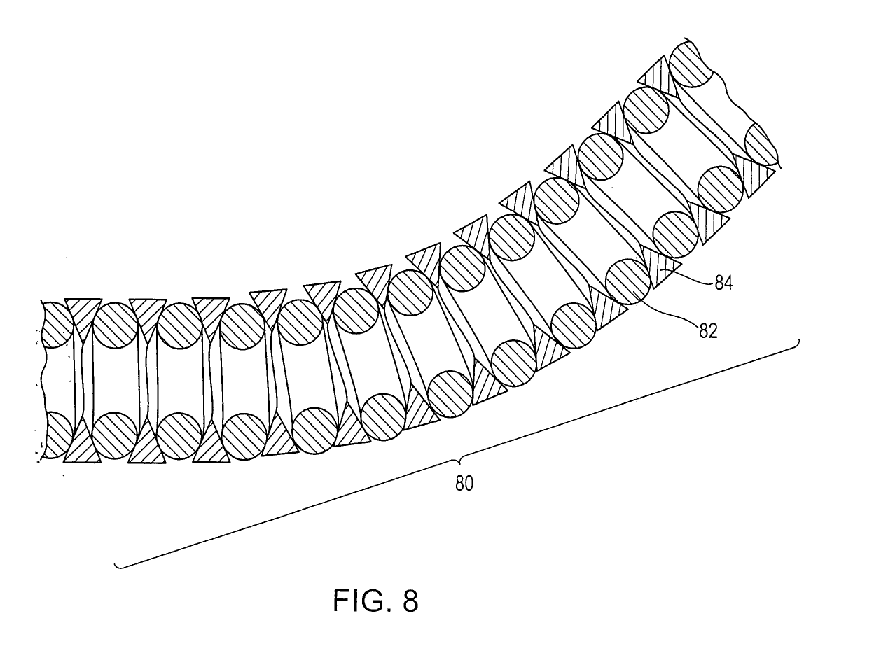

FIG. 8 illustrates a cross-sectional view of a semi-rigid portion 80 that is in a bent orientation. The semi-rigid portion 80 is formed from a first element 82 and a second element 84. The above-mentioned frictional relationship is better explained with the bent cross-section diagram in FIG. 8. The first element 82 has a tendency to remain in a straight condition, like a spring, with a consistent separation between adjacent circular sections. The second element 84, however, when bent, has a tendency to remain at a consistent diameter. The shape of the second element 84, in this example, forces the separation of adjacent sections of the first element 82 under bending. At the same time, the adjacent sections of the second element 84 are forced inward on an outside diameter of the bend and outward on the inside diameter of the bend. The forces between the two elements 82 and 84, though antagonistic, create the necessary friction between the two elements 82 and 84 to maintain the desired bend position of the flexible portion 80. When straightening the device, the reverse of the above actions occurs between the first and second elements 82 and 84.

It will be appreciated that the various of the above-disclosed and other features and functions, or alternatives thereof, may be desirably combined into many other different devices or applications. Also, various presently unforeseen or unanticipated alternatives, modifications, variations or improvements therein may be subsequently made by those skilled in the art which are also intended to be encompassed by the following claims.

What is claimed is:

1. A flexible-shaft surgical instrument, comprising:
at least one rigid tube portion;
at least one semi-rigid tube portion connected to the at least one rigid tube portion, the semi-rigid tube portion comprising a plurality of wound elements, each of the wound elements are interleaved together about a central axis such that any adjacent portions of each of the plurality of wound elements experience a degree of friction between them, the at least one semi-rigid tube portion being configured to be bent to a predetermined angle and returned to a pre-bent orientation repeatably and without plastic deformation; and
at least one surgical device,
wherein an internal diameter of the at least one rigid tube portion and the at least one semi-rigid tube portion accommodates the at least one surgical device, and access for the at least one surgical device to a target site in a patient's body is provided through the surgical instrument via at least one opening in or adjacent to a distal end of the surgical instrument.

2. The surgical instrument of claim 1, wherein, in an axial cross-section through the central axis, the plurality of wound elements have different cross-sectional shapes.

3. The surgical instrument of claim 1, wherein the at least one semi-rigid tube portion is connected to a distal end of the at least one rigid tube portion.

4. The surgical instrument of claim 1, wherein the semi-rigid tube portion is of a length long enough such that the at least one semi-rigid portion may be bent between an angle of 0° and 275°.

5. The surgical instrument of claim 1, wherein the semi-rigid tube portion is of a substantially equal diameter across its length.

6. The surgical instrument of claim 1, wherein the semi-rigid tube portion varies in diameter across its length.

7. The surgical instrument of claim 1, wherein the at least one rigid tube portion comprises at least two rigid tube portions.

8. The surgical instrument of claim 1, wherein the at least one semi-rigid tube portion comprises at least two semi-rigid tube portions,
wherein first and second of the at least two semi-rigid tube portions are configured to be orientated in the same or varying planes.

9. The surgical instrument of claim 1, wherein, in an axial cross-section through the central axis, a cross-section of at least one wound element of the semi-rigid tube portion is circular.

10. The surgical instrument of claim 1, wherein, in an axial cross-section through the central axis, a cross-section of at least one wound element of the semi-rigid tube portion is triangular.

11. The surgical instrument of claim 1, further comprising a protective sheath covering at least the at least one semi-rigid portion.

12. The surgical instrument of claim 1, wherein a material for the semi-rigid tube portion provides a friction between the plurality of wound elements of the wound portion so that the semi-rigid portion holds a predetermined bend orientation.

13. The surgical instrument of claim 1, wherein the semi-rigid tube portion comprises a material that is non-conductive.

14. The surgical instrument of claim 1, wherein the semi-rigid tube portion comprises a material that is temperature resistant.

15. The surgical instrument of claim 1, wherein the semi-rigid tube portion comprises a material that is molded.

16. The surgical instrument of claim 1, wherein the semi-rigid tube portion comprises a material that is biocompatible.

17. The surgical instrument of claim 1, wherein the semi-rigid tube portion comprises a material that is not degraded by chemicals employed in a surgical procedure.

18. The surgical instrument of claim 1, wherein the at least one surgical device comprises at least one of a shaver, light, vacuum, suction lumen, camera, observation device, microdebrider, or electro-surgical probe.

19. The surgical instrument of claim 1, wherein the semi-rigid tube portion is formed from a metal material.

20. The surgical instrument of claim 1, wherein the plurality of wound elements are formed of a same material.
